(19) United States
(12) Patent Application Publication
(10) Pub. No.: US 2022/0165401 A1
(43) Pub. Date: May 26, 2022

(54) SYSTEM AND METHOD FOR SCHEDULING APPOINTMENTS IN THE FIELD OF HEALTHCARE

(71) Applicant: Upractice Dotcom LLC, El paso, TX (US)

(72) Inventor: Aubrey LEVITT, Houston, TX (US)

(73) Assignee: Upractice Dotcom LLC, El paso, TX (US)

(21) Appl. No.: 17/534,639

(22) Filed: Nov. 24, 2021

Related U.S. Application Data
(60) Provisional application No. 63/118,257, filed on Nov. 25, 2020.

(51) Int. Cl. G16H 40/20; G16H 15/00

(52) U.S. Cl. CPC G16H 40/20; G16H 15/00

(57) ABSTRACT

A method is provided to access one or more medical codes associated with a patient in receiving medical care. A standard of care associated with the one or more medical codes is determined. An unscheduled appointment associated with the medical care for the patient is identified based on the medical codes and the standard of care. The unscheduled appointment is scheduled.

### CROSS-REFERENCE TO RELATED APPLICATION

This application claims the benefit of U.S. Provisional Patent Application No. 63/118,257, filed in the U.S. Patent and Trademark Office on Nov. 25, 2020, which is incorporated herein by reference in its entirety for all purposes.

### TECHNICAL FIELD

The present technology pertains to scheduling appointments, and more specifically to the scheduling of unscheduled follow-up healthcare appointments.

### BACKGROUND

The healthcare industry is slowly beginning to automate their systems and procedures to evolve into an integrated, user-friendly computerized solution. Proper automation and recordkeeping helps practices implement standards of care. Different data management systems, such as electronic health records, scheduling information, and billing information should be stored, tracked, and maintained in an integrated fashion and delivered to the healthcare provider or their patients.

Most healthcare practices rely on scheduled appointments. After seeing a patient, a healthcare provider may recommend a return appointment for some time in the future. Sometimes, the return appointment is too far into the future. A notice may be sent to the patient reminding the patient to call to schedule an appointment. It is important to identify the patients that are overdue for healthcare service to meet the standard of care in the healthcare industry. By helping to meet the standard of care, insurance companies are more likely to pay for rendered procedures and services, thereby reducing costs for patients and increase doctor revenue. Therefore, it is necessary to have a platform that integrates different data management systems in order to track and identify patients that are overdue for an appointment.

### BRIEF DESCRIPTION OF THE DRAWINGS

In order to describe the manner in which the above-recited and other advantages and features of the disclosure can be obtained, a more particular description of the principles briefly described above will be rendered by reference to specific embodiments thereof which are illustrated in the appended drawings. Understanding that these drawings depict only exemplary embodiments of the disclosure and are not therefore to be considered to be limiting of its scope, the principles herein are described and explained with additional specificity and detail through the use of the accompanying drawings in which:

FIG. 1 illustrates an example schematic diagram of a healthcare system;

FIG. 2 illustrates an example flowchart for scheduling healthcare appointments;

FIG. 3 illustrates an example screenshot of a graphical user interface to review and schedule healthcare appointments;

FIG. 4 illustrates an example flowchart for scheduling healthcare appointments;

FIG. 5 illustrates an example flowchart for scheduling healthcare appointments; and

FIG. 6 illustrates a computing system architecture, according to various embodiments of the present disclosure.

### DETAILED DESCRIPTION

Various embodiments of the disclosure are discussed in detail below. While specific implementations are discussed, it should be understood that this is done for illustration purposes only. A person skilled in the relevant art will recognize that other components and configurations may be used without parting from the spirit and scope of the disclosure. Thus, the following description and drawings are illustrative and are not to be construed as limiting. Numerous specific details are described to provide a thorough understanding of the disclosure. However, in certain instances, well-known or conventional details are not described in order to avoid obscuring the description. References to one or an embodiment in the present disclosure can be references to the same embodiment or any embodiment; and, such references mean at least one of the embodiments.

Reference to “one embodiment” or “an embodiment” means that a particular feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others.

The terms used in this specification generally have their ordinary meanings in the art, within the context of the disclosure, and in the specific context where each term is used. Alternative language and synonyms may be used for any one or more of the terms discussed herein, and no special significance should be placed upon whether or not a term is elaborated or discussed herein. In some cases, synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any example term. Likewise, the disclosure is not limited to various embodiments given in this specification.

Without intent to limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, technical and scientific terms used herein have the meaning as commonly understood by one of ordinary skill in the art to which this disclosure pertains. In the case of conflict, the present document, including definitions will control.

Additional features and advantages of the disclosure will be set forth in the description which follows, and in part will be obvious from the description, or can be learned by practice of the herein disclosed principles. The features and advantages of the disclosure can be realized and obtained by means of the instruments and combinations particularly pointed out in the appended claims. These and other features of the disclosure will become more fully apparent from the following description and appended claims, or can be learned by the practice of the principles set forth herein.

Various embodiments of the subject technology relate to scheduling appointments, and more specifically to the scheduling of follow-up healthcare appointments that were unscheduled.

In various embodiments, follow-up appointments that have not been booked are captured based on standards of care as they relate to medical codes by specialty. This helps ensure that follow-up appointments are booked, thereby improving the overall standard of care for patients. Data tables may be used that correlate CPT codes, ICD-10 codes, specialties, standards of care, doctor's information, patient information, patient visits that were billed, and/or messages that will be sent to the patient to facilitate booking of the follow-up appointment. Patients who see their providers regularly have better relationships, increased satisfaction, and improved outcomes.

Conventionally, the process to determine unscheduled and/or missed appointments is a manual process. Periodically (e.g., 3-4 months), the provider may print out a list to see which patients have not scheduled and attended follow up appointments. This is a tedious process, and many patients and appointments are missed.

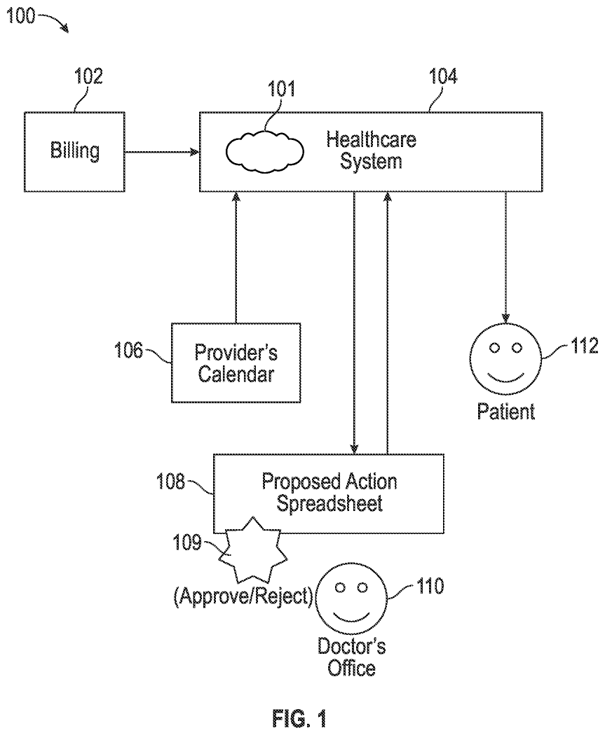

FIG. 1 illustrates a diagram illustrating an example network environment 100 utilizing a healthcare system 104. The healthcare system 104 may be connected to different systems (e.g., billing system 102, provider's calendar 106, proposed action spreadsheet 108) via a network 101. The network 101 can include, for example, any one or more of a cellular network, a satellite network, a personal area network (PAN), a local area network (LAN), a wide area network (WAN), a broadband network (BBN), the Internet, Bluetooth, radio frequency identification (RFID), and/or the like. Further, the network 101 can include, but is not limited to, any one or more of the following network topologies, including a bus network, a star network, a ring network, a mesh network, a star-bus network, tree or hierarchical network, and the like.

In some examples, patient data may be stored in accordance with regulations, such as HIPAA. As such, data stored in the healthcare system 104 may be segmented such that data that may identify a patient may be removed from medical data. In such an example, minimal patient data (e.g., patient ID, first and last name, email, date of birth, and/or address) may be stored in a HIPAA compliant data store, and data relating to prior medication purchases, medical records from healthcare organizations, medical condition, biometric data, genetic data, health literacy data, telehealth consultation data, wearable device data, medication adherence data, family composition data, supplement and vitamin use, retail purchase data, condition predictive algorithm data, health condition status, and/or chat bot data may be stored in an anonymous data store. The data in the anonymous data store may be keyed by the user ID which represents a unique alpha-numeric or numeric code (e.g., 339fj3-33d4-fkfkf-33e3) that yields no identifying information on its own.

A billing system 102 may communicate with the healthcare system 102 to provide medical codes input by a provider (e.g., doctor, hospital network, private clinic, etc.) after a patient's visit. The medical codes may reflect the service(s) that the provider performed and/or diagnosed during the patient's visit. In some examples, the medical codes may include international classification of diseases (ICD) codes. In some examples, the medical codes may include ICD-10 codes. In some examples, the medical codes may include current procedural terminology (CPT) codes. The medical codes may be pushed to the healthcare system 104 from billing system 102 upon entry into an electronic medical record (EMR) system. In some examples, the medical codes may be pushed to the healthcare system 104 from billing system 102 at a predetermined scheduled time. In some examples, the healthcare system 104 may retrieve the medical codes from billing system 102.

The provider's calendar 106 can include the scheduled appointments for patients. The healthcare system 104 may access the provider's calendar 106 to determine what appointments were scheduled and/or attended.

A proposed action spreadsheet 108 may include the patient information (e.g., patient name, date of birth, address, etc.), diagnosis based on the medical codes, risk, suggested date for the suggested appointment based on the medical codes, notes for the suggested appointment, and/or actions (e.g., approve and/or reject sending out appointment). The healthcare system 104 may populate the proposed action spreadsheet 108. The doctor's office or provider's office 110 may review the proposed action spreadsheet 108 and determine whether to approve and/or reject 109 suggested appointments or suggested actions. The action 109 by the provider's office 110 in response to the action items in the proposed action spreadsheet 108 may be received by the healthcare system 104. In some examples, the healthcare system 104 may contact the patient 112 based on the response to the action items in the proposed action spreadsheet 108. For example, when the doctor's office 110 approves 109 the suggested appointment in the proposed action spreadsheet 108, the healthcare system 104 may contact the patient 112 with the suggested appointment and/or a reminder to contact the provider's office 110 to schedule an appointment. The healthcare system 104 may contact the patient 112 via various mechanisms, for example text message, email, app, or any other suitable mechanism. In some examples, the mechanism to contact the patient 112 may be predetermined by the patient 112 and/or the provider's office 110.

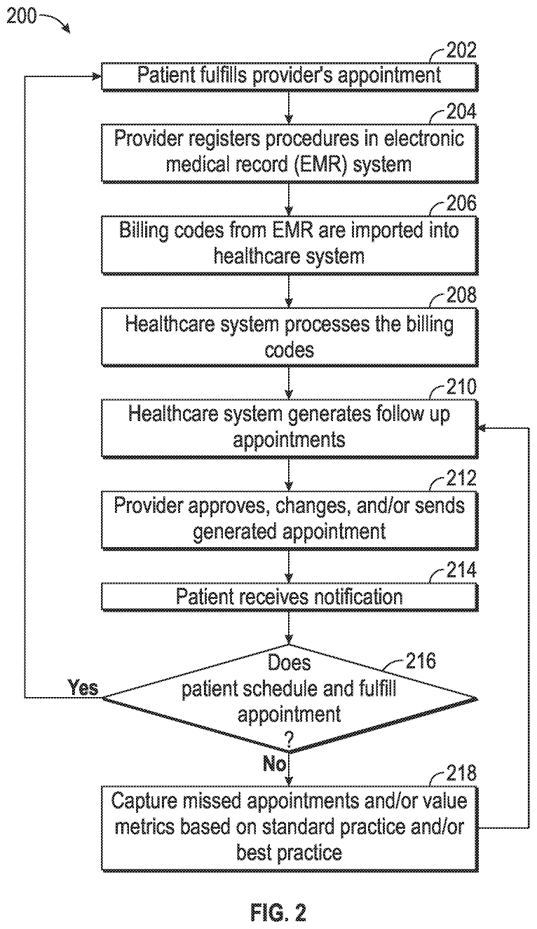

Referring to FIG. 2, a flowchart for scheduling healthcare appointments is presented in accordance with an example embodiment. The method 200 is provided by way of example, as there are a variety of ways to carry out the method. The method 200 described below can be carried out using the configurations and/or features illustrated and discussed illustrated in FIGS. 1 and 3-6, for example, and various elements of these figures are referenced in explaining example method 200. Each block shown in FIG. 2 represents one or more processes, methods or subroutines, carried out in the example method 200. Furthermore, the illustrated order of blocks is illustrative only and the order of the blocks can change according to the present disclosure. Additional blocks may be added or fewer blocks may be utilized, without departing from this disclosure. The example method 200 can begin at block 202.

At block 202, the patient fulfills a provider's appointment. During the appointment, the provider may diagnose or conduct a procedure to address the patient's issue(s). Based on the actions and/or determinations during the appointment, at block 204, the provider registers the procedures and/or determinations in a billing system, for example an electronic medical record (EMR) system. The registering of procedures and/or determinations are entered in the EMR system by medical codes. The medical codes may reflect what procedures and/or determinations were conducted during the appointment. The medical codes may include ICD codes, for example ICD-10 codes. In some examples, the medical codes may include CPT codes. For example, an ICD-10 code C67.0 may be entered into the EMR system which reflects a diagnosis of bladder neoplasm—trigone.

At block 206, the medical codes from the EMR system are imported into the healthcare system. In some examples, the medical codes may be pushed to the healthcare system from the EMR system at a predetermined scheduled time. In some examples, the healthcare system may retrieve the medical codes from the EMR system.

At block 208, the healthcare system processes the medical codes. The healthcare system can determine, based on the medical codes, what the diagnosis is, and when the next follow up appointment should be. In some examples, the healthcare system may include a database which includes one or more references such as textbooks, articles, insurance plans, best practices, standards of care, etc. The healthcare system may make the determinations based on one or more references in the database. By utilizing one or more references, the healthcare system may determine the most accurate course of actions based on the standard of care and/or the best practices. The healthcare system may process the medical codes by analyzing the medical codes, determine the diagnosis, determine the standard of care and/or best practices, and determine the next follow up appointment based on the references. Accordingly, during processing of the medical codes, the standard of care associated with the medical codes is determined. While the term standard of care is utilized, in some examples, best practice may be utilized instead of or in addition to the standard of care. The standard of care is published practice while the best practice is industry standard. For example, if standard of care is not available, then best practice is determined. The insurance plans may assist in determining whether the insurance would cover the follow up appointment, and what course of action is approved by the insurance plan. This helps ensure that the patient is covered by their insurance plan, which would improve the adherence to the treatment plan by the patient. In some examples, the healthcare system may determine the risk factor for the patient based on the medical codes. The risk factor may reflect the severity of the diagnosis to the patient, and guide in determining the importance of the follow up appointment. If the risk factor is high, the follow up appointment is much more important, or the patient may suffer great harm if the follow up appointment is missed or not scheduled. For example, the ICD-10 code C67.0 may be determined to reflect a diagnosis of bladder neoplasm—trigone. The healthcare system, in processing the medical code, may refer to the database. For example, the reference utilized may be the textbook Bladder Cancer 1^{st} Edition. The healthcare system may process the medical code to determine that the follow up appointment should be scheduled and fulfilled in three months from the original appointment. Also, the healthcare system may determine that the risk factor is very high, for example 5 out of a scale of 5. In other examples, different risk factors may be used such as a scale out of 10, a scale out of 100, or any other suitable scale.

At block 210, the healthcare system generates one or more follow up appointments based on the standard of care. For example, the healthcare system may generate a follow up appointment for three months based on the standard of care for bladder neoplasm—trigone. The healthcare system may access the provider's calendar to determine whether the follow up appointment has been scheduled, whether the follow up appointment has been fulfilled, and/or how many past appointments have been fulfilled by the patient. If the follow up appointment has already been scheduled on the calendar, then the healthcare system may not send a follow up appointment. However, if no appointment has been scheduled in a range of time around the determined follow up appointment time, the healthcare system may send a follow up appointment. Also, if the patient has fulfilled many past appointments, the risk factor for the patient to not attend is lowered. However, if the patient regularly missed appointments or did not schedule follow up appointments, the risk factor may be raised.

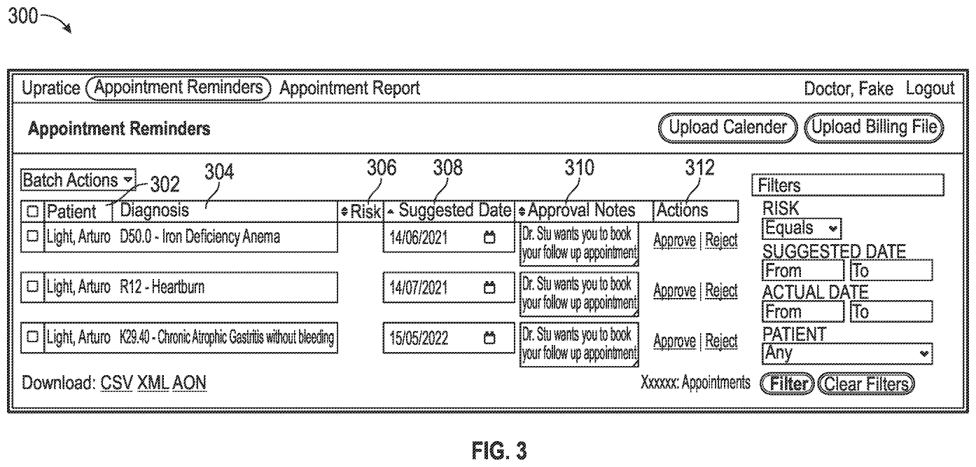

At block 212, the provider may approve, change, and/or send the generated appointment. For example, the healthcare system may generate and/or populate a proposed action spreadsheet. The provider may approve, change, and/or send the generated appointment via the proposed action spreadsheet. An example of a proposed action spreadsheet is shown in FIG. 3. The proposed action spreadsheet 300 can include a patient's name 302, diagnosis and/or medical codes 304, risk factor 306, suggested date for follow up appointment 308, notes 310, and actions 312. For example, the patient name 302 may be Arturo Light. The diagnosis may include an iron deficiency anemia based on the medical code D50.0. The risk factor in this example is not included, but the risk factor may reflect the severity of the diagnosis and importance of having a follow up appointment. The suggested date for a follow up appointment may be Jun. 14, 2021. The suggested date reflects a date within a range of time for the follow up appointment that is available in the provider's calendar. In some examples, the provider may adjust the date. In some examples, the proposed action spreadsheet may reflect the range of dates that are acceptable for a follow up appointment, for example a two-week time range around the 3 month follow up date. The notes 310 may reflect notes that can be sent to the patient with the follow up appointment. The notes 310 may also reflect notes for the provider for a reminder to reach out to the patient regarding a follow up appointment or any further discussion. The provider may adjust the notes. The actions 312 may include approve or reject. If the action 312 chosen is to approve, the follow up appointment may be sent to the patient. In some examples, the healthcare system may contact the patient, for example via text, phone call, email, app, and/or any other suitable mechanism. The contact attempt with the follow up appointment and/or any response from the patient may logged by the healthcare system. If the action 312 chosen if reject, the follow up appointment is not sent out. This action may also be logged by the healthcare system.

At block 214, the patient receives the notification. At block 216, the healthcare system may determine whether the patient scheduled and/or fulfilled the follow up appointment. For example, the healthcare system may access the provider's calendar to determine whether the follow up appointment was scheduled and/or fulfilled. If the patient scheduled and fulfilled the follow up appointment, new medical codes from that appointment may be entered, and the process returns to block 202.

If the patient did not schedule and/or fulfill the appointment, at block 218, the healthcare system may capture and/or log the missed appointment(s) and/or value metrics based on the standard practice and/or best practice. The process may proceed to block 210, and the healthcare system may generate another follow up appointment to try to schedule the patient once again.

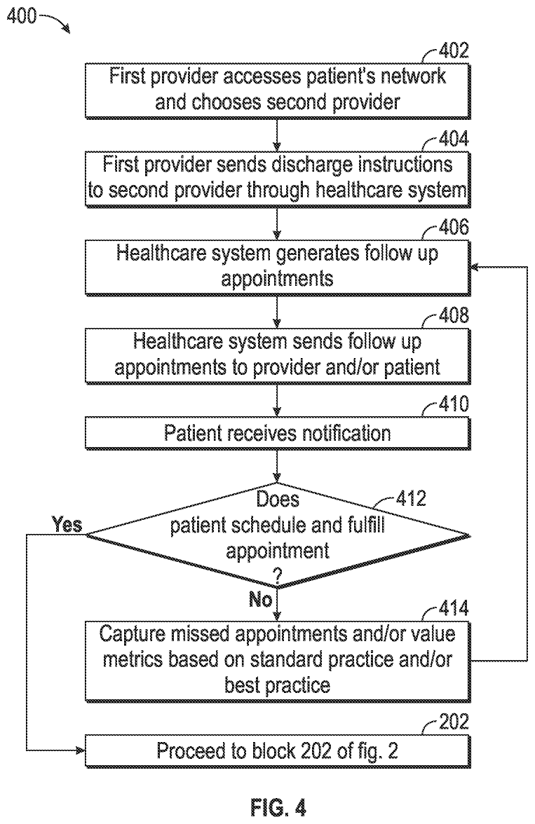

Referring to FIG. 4, a flowchart for scheduling healthcare appointments is presented in accordance with an example embodiment. The method 400 is provided by way of example, as there are a variety of ways to carry out the method. The method 400 described below can be carried out using the configurations and/or features illustrated and discussed in FIGS. 1-3 and 5-6, for example, and various elements of these figures are referenced in explaining example method 400. Each block shown in FIG. 4 represents one or more processes, methods or subroutines, carried out in the example method 400. Furthermore, the illustrated order of blocks is illustrative only and the order of the blocks can change according to the present disclosure. Additional blocks may be added or fewer blocks may be utilized, without departing from this disclosure. The example method 400 can begin at block 402.

The method 400 is similar to the method 200 in FIG. 2, but also integrating communication between different providers. For example, a patient may go to the hospital and have an emergency appointment. Conventionally, the patient gets a diagnosis and/or treatment at the hospital, but no communication with another provider (e.g., specialist, family provider, etc.). The onus of the follow up is placed on the patient. Accordingly, conventionally many follow up appointments that are needed to fully treat the patient are missed, which may harm the patient in the long term.

At block 402, the first provider accesses the patient's network and chooses a second provider. The first provider, for example, may be a hospital. The patient's network, for example, may be determined by accessing the insurance network to ensure that the patient is staying in network. This can benefit the patient in lowering costs by staying in network as well as helping ensure the insurance will cover the next appointment(s). The second provider, for example, may be a urology specialist. In some examples, the first provider may access the patient's network by scanning a code, for example a QR code and/or a barcode, provided on the insurance card. In some examples, the first provider may access the patient's network by entering a code, for example provided on the insurance card. In some examples, the first provider may communicate with the patient the different second providers available, and the patient can help select the second provider.

At block 404, once the second provider is chosen, the first provider can send discharge instructions to the second provider through the healthcare system.

At block 406, the healthcare system can process the discharge instructions and/or any medical codes and generate follow up appointments for the patient at the second provider. In some examples, this can be similar to block 208 of FIG. 2.

At block 408, the healthcare system sends the follow up appointment(s) to the second provider and/or the patient. In some examples, this can be similar to blocks 210 and/or 212 of FIG. 2.

At block 410, the patient receives the notification. In some examples, this can be similar to block 214 of FIG. 2.

At block 412, similar to block 216 of FIG. 2, a determination is made whether the patient schedules and fulfills the appointment. If the patient does schedule and fulfill the appointment, the process proceeds to block 202 of FIG. 2. If the patient does not schedule and/or fulfill the appointment, the healthcare system may capture and/or log the missed appointment(s) and/or value metrics based on the standard practice and/or best practice. The process may proceed to block 407, and the healthcare system may generate another follow up appointment to try to schedule the patient once again.

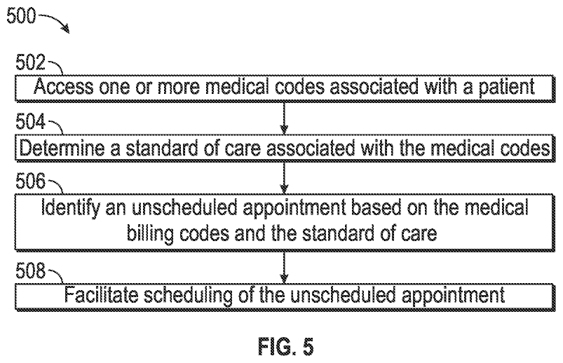

Referring to FIG. 5, a flowchart for scheduling healthcare appointments is presented in accordance with an example embodiment. The method 500 is provided by way of example, as there are a variety of ways to carry out the method. The method 500 described below can be carried out using the configurations and/or features illustrated and discussed in FIGS. 1-4 and 6, for example, and various elements of these figures are referenced in explaining example method 500. Each block shown in FIG. 5 represents one or more processes, methods or subroutines, carried out in the example method 500. Furthermore, the illustrated order of blocks is illustrative only and the order of the blocks can change according to the present disclosure. Additional blocks may be added or fewer blocks may be utilized, without departing from this disclosure. The example method 500 can begin at block 502.

At block 502, one or more medical codes associated with a patient in receiving medical care are accessed. The one or more medical codes may include ICD codes, for example ICD-10 codes. In some examples, the one or more medical codes may include CPT codes. At block 504, a standard of care associated with the one or more medical codes is determined. While the term standard of care is utilized, in some examples, best practice may be utilized instead of or in addition to the standard of care. The standard of care is published practice while the best practice is industry standard. For example, if standard of care is not available, then best practice is determined. At block 506, an unscheduled appointment associated with the medical care for the patient based on the one or more medical billing codes and the standard of care is identified. At block 508, scheduling of the unscheduled appointment is facilitated. For example, the unscheduled appointment may be scheduled with a care provider associated with rendering the medical care. As another example, the unscheduled appointment may also be scheduled with the patient. As another example, the scheduling of the unscheduled appointment between the care provider associated with rendering the medical care and the patient may be coordinated.

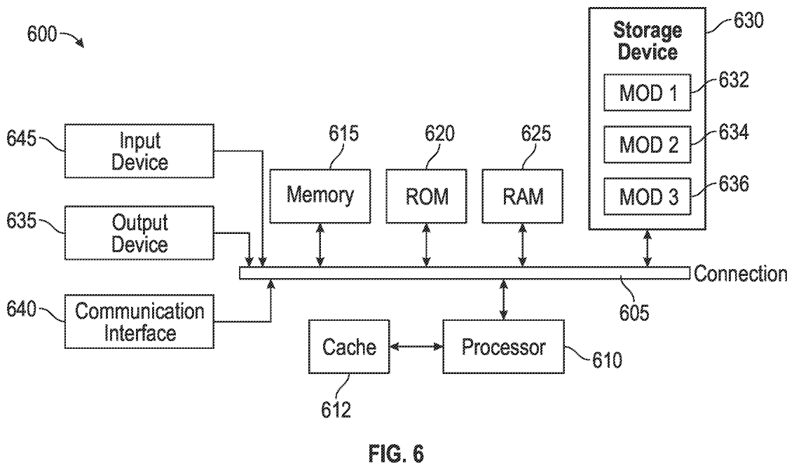

FIG. 6 illustrates a computing system architecture 600. The system architecture 600 architecture may be architected with access layers that ensure HIPAA (Health Insurance Portability and Accountability Act) compliance and/or compliance with the HITECH (Health Information Technology for Economic and Clinical Health) Act. The system 600 may have functionality to assist in compliance with the HIPAA Privacy Regulations, such as consent tracking and disclosure logging functionalities. The system 600 also supports EDI standards, including transactions, code sets, and identifiers. The components of the system are in electrical communication with each other using a connection 605, such as a bus. Exemplary system 600 includes a processing unit (CPU or processor) 610 and a system connection 605 that couples various system components including the system memory 615, such as read only memory (ROM) 620 and random access memory (RAM) 625, to the processor 610. The system 600 can include a cache of high-speed memory connected directly with, in close proximity to, or integrated as part of the processor 610. The system 600 can copy data from the memory 615 and/or the storage device 630 to the cache 612 for quick access by the processor 610. In this way, the cache can provide a performance boost that avoids processor 610 delays while waiting for data. These and other modules can control or be configured to control the processor 610 to perform various actions. Other system memory 615 may be available for use as well. The memory 615 can include multiple different types of memory with different performance characteristics. The processor 610 can include any general purpose processor and a hardware or software service, such as service 1 632, service 2 634, and service 3 636 stored in storage device 630, configured to control the processor 610 as well as a special-purpose processor where software instructions are incorporated into the actual processor design. The processor 610 may be a completely self-contained computing system, containing multiple cores or processors, a bus, memory controller, cache, etc. A multi-core processor may be symmetric or asymmetric.

To enable user interaction with the computing device 600, an input device 645 can represent any number of input mechanisms, such as a microphone for speech, a touch-sensitive screen for gesture or graphical input, keyboard, mouse, motion input, speech and so forth. An output device 635 can also be one or more of a number of output mechanisms known to those of skill in the art. In some instances, multimodal systems can enable a user to provide multiple types of input to communicate with the computing device 600. The communications interface 640 can generally govern and manage the user input and system output. There is no restriction on operating on any particular hardware arrangement and therefore the basic features here may easily be substituted for improved hardware or firmware arrangements as they are developed.

Storage device 630 is a non-volatile memory and can be a hard disk or other types of computer readable media which can store data that are accessible by a computer, such as magnetic cassettes, flash memory cards, solid state memory devices, digital versatile disks, cartridges, random access memories (RAMs) 325, read only memory (ROM) 320, and hybrids thereof.

The storage device 630 can include services 632, 634, 636 for controlling the processor 610. Other hardware or software modules are contemplated. The storage device 630 can be connected to the system connection 605. In one aspect, a hardware module that performs a particular function can include the software component stored in a computer-readable medium in connection with the necessary hardware components, such as the processor 610, connection 605, output device 635, and so forth, to carry out the function.

For clarity of explanation, in some instances the present technology may be presented as including individual functional blocks including functional blocks comprising devices, device components, steps or routines in a method embodied in software, or combinations of hardware and software.

In some embodiments the computer-readable storage devices, mediums, and memories can include a cable or wireless signal containing a bit stream and the like. However, when mentioned, non-transitory computer-readable storage media expressly exclude media such as energy, carrier signals, electromagnetic waves, and signals per se.

Methods according to the above-described examples can be implemented using computer-executable instructions that are stored or otherwise available from computer readable media. Such instructions can comprise, for example, instructions and data that cause or otherwise configure a general purpose computer, special purpose computer, or special purpose processing device to perform a certain function or group of functions. Portions of computer resources used can be accessible over a network. The computer executable instructions may be, for example, binaries, intermediate format instructions such as assembly language, firmware, or source code. Examples of computer-readable media that may be used to store instructions, information used, and/or information created during methods according to described examples include magnetic or optical disks, flash memory, USB devices provided with non-volatile memory, networked storage devices, and so on.

Devices implementing methods according to these disclosures can comprise hardware, firmware and/or software, and can take any of a variety of form factors. Typical examples of such form factors include laptops, smart phones, small form factor personal computers, personal digital assistants, rackmount devices, standalone devices, and so on. Functionality described herein also can be embodied in peripherals or add-in cards. Such functionality can also be implemented on a circuit board among different chips or different processes executing in a single device, by way of further example.

The instructions, media for conveying such instructions, computing resources for executing them, and other structures for supporting such computing resources are means for providing the functions described in these disclosures.

Given the mass amounts of data that may be processed, in some examples, machine-learning based classification techniques can vary depending on the desired implementation. Utilizing machine-learning based classification techniques makes the system run smoothly and efficiently in contrast to the conventionally manual process that leads to many missed appointments and lost patients. For example, machine-learning classification schemes can utilize one or more of the following, alone or in combination: hidden Markov models, recurrent neural networks (RNNs), convolutional neural networks (CNNs); Deep Learning networks, Bayesian symbolic methods, general adversarial networks (GANs), support vector machines, image registration methods, and/or applicable rule-based systems. Where regression algorithms are used, they can include but are not limited to: a Stochastic Gradient Descent Regressors, and/or Passive Aggressive Regressors, etc.

Machine learning classification models can also be based on clustering algorithms (e.g., a Mini-batch K-means clustering algorithm), a recommendation algorithm (e.g., a Miniwise Hashing algorithm, or Euclidean Locality-Sensitive Hashing (LSH) algorithm), and/or an anomaly detection algorithm, such as a Local outlier factor. Additionally, machine-learning models can employ a dimensionality reduction approach, such as, one or more of: a Mini-batch Dictionary Learning algorithm, an Incremental Principal Component Analysis (PCA) algorithm, a Latent Dirichlet Allocation algorithm, and/or a Mini-batch K-means algorithm, etc.

Although a variety of examples and other information was used to explain aspects within the scope of the appended claims, no limitation of the claims should be implied based on particular features or arrangements in such examples, as one of ordinary skill would be able to use these examples to derive a wide variety of implementations. Further and although some subject matter may have been described in language specific to examples of structural features and/or method steps, it is to be understood that the subject matter defined in the appended claims is not necessarily limited to these described features or acts. For example, such functionality can be distributed differently or performed in components other than those identified herein. Rather, the described features and steps are disclosed as examples of components of systems and methods within the scope of the appended claims.

What is claimed is:

1. A method comprising:
accessing one or more medical codes associated with a patient in receiving medical care;
determining a standard of care associated with the one or more medical codes;
identifying an unscheduled appointment associated with the medical care for the patient based on the one or more medical codes and the standard of care; and
facilitating scheduling of the unscheduled appointment.

2. The method of claim 1, wherein facilitating scheduling of the unscheduled appointment includes scheduling the unscheduled appointment with a care provider associated with rendering the medical care.

3. The method of claim 1, wherein facilitating scheduling of the unscheduled appointment includes scheduling the unscheduled appointment with the patient.

4. The method of claim 1, wherein facilitating scheduling of the unscheduled appointment includes coordinating scheduling of the unscheduled appointment between a care provider associated with rendering the medical care and the patient.

5. The method of claim 1, wherein the one or more medical codes include international classification of diseases (ICD) codes.

6. The method of claim 5, wherein the one or more medical codes include ICD-10 codes.

7. The method of claim 1, wherein the one or more medical codes include current procedural terminology (CPT) codes.

8. A system comprising:
a processor that executes instructions stored in memory, wherein the processor executes the instructions to:
access one or more medical codes associated with a patient in receiving medical care,
determine a standard of care associated with the one or more medical codes,
identify an unscheduled appointment associated with the medical care for the patient based on the one or more medical codes and the standard of care; and
a user interface that facilitates scheduling of the unscheduled appointment.

9. The system of claim 8, wherein the user interface facilitates scheduling of the unscheduled appointment by scheduling the unscheduled appointment with a care provider associated with rendering the medical care.

10. The system of claim 8, wherein the user interface facilitates scheduling of the unscheduled appointment by scheduling the unscheduled appointment with the patient.

11. The system of claim 8, wherein the user interface facilitates scheduling of the unscheduled appointment by coordinating scheduling of the unscheduled appointment between a care provider associated with rendering the medical care and the patient.

12. The system of claim 8, wherein the one or more medical codes include international classification of diseases (ICD) codes.

13. The system of claim 12, wherein the one or more medical codes include ICD-10 codes.

14. The system of claim 8, wherein the one or more medical codes include current procedural terminology (CPT) codes.

15. A non-transitory, computer-readable storage medium, having instructions encoded thereon, the instructions executable by a processor to perform a method, the method comprising:
accessing one or more medical codes associated with a patient in receiving medical care;
determining a standard of care associated with the one or more medical codes;
identifying an unscheduled appointment associated with the medical care for the patient based on the one or more medical codes and the standard of care; and
facilitating scheduling of the unscheduled appointment.
